(19) United States
(12) Patent Application Publication
(10) Pub. No.: US 2003/0083535 A1
(43) Pub. Date: May 1, 2003

(54) Circulating Catalyst system and method for conversion of light hydrocarbons to aromatics

(75) Inventors: Harold A. Wright, Ponca City, OK (US); Todd H. Harkins, Ponca City, OK (US); Doug S. Jack, Ponca City, OK (US); Ajoy P. Raje, Stillwater, OK (US)

Correspondence Address: DAVID W. WESTPHAL, CONOCO PHILLIPS, P.O. BOX 4783, HOUSTON, TX, 77210-4783, US

(73) Assignee: Conoco Inc., Houston, TX (US)

(21) Appl. No.: 10/139,485

(22) Filed: May 6, 2002

Related U.S. Application Data
(60) Provisional application No. 60/299,545, filed on Jun. 20, 2001.

(51) Int. Cl. C07C 15/00; B01J 8/18

(52) U.S. Cl. 585/943; 585/407; 585/412; 422/139; 422/141; 422/144; 422/145

(57) ABSTRACT

The present invention features a system and method for circulating catalyst between a reactor system and a regenerator system. A circulating catalyst system includes a reactor system, a regenerator system, and a distribution unit. The reactor system and regenerator system are adapted to exchange catalyst. The regeneration system preferably includes a regeneration zone adapted for the contact of catalyst with a regeneration gas. The system and method are adapted so that more than one regeneration gas may contact catalyst. The distribution unit is adapted to control the percentage of catalyst contacting each regeneration gas. Thus, the distribution unit is adapted to select the percentages so as to maintain the reactor system and regeneration system under a heat balance regime. Heat is preferably transferred from the regenerator system to the reactor system by an exchange of catalyst.

### CROSS-REFERENCE TO RELATED APPLICATIONS

The present application claims the benefit of priority from U.S. Provisional Application Serial No. 60/299,545, filed Jun. 20, 2001, entitled “Circulating Catalyst System and Method for Conversion of Light Hydrocarbons to Aromatics”. Further, the present application is related to U.S. aplication Ser. No. 09/916,469, filed Jul. 27, 2001, which claims the benefit of priority from U.S. Provisional Application Serial No. 60/221,082, each entitled “Catalyst and Process for Aromatic Hydrocarbons Production from Methane”. Each of the above-listed applications is hereby incorporated herein by reference.

### STATEMENT REGARDING FEDERALLY SPONSORED RESEARCH OR DEVELOPMENT

Not applicable.

### FIELD OF THE INVENTION

The present invention relates to a system and method for catalytically converting a light hydrocarbon feed to an aromatic product. In particular, the present invention relates to a system and method for circulating catalyst between a reactor system and a regenerator system. More particularly, the present invention relates to a system and method for controlling the heat generated by the regeneration of the catalyst and transferring the heat to the reactor system.

### BACKGROUND OF THE INVENTION

Natural gas is an abundant fossil fuel resource. Recent estimates place worldwide natural gas reserves at about 35×10¹⁴ standard cubic feet, corresponding to the energy equivalent of about 637 billion barrels of oil. However, a significant portion of the known natural gas reserves is associated with fields found in remote regions that are difficult to access. For many of these remote fields, transporting the gas to potential users is not economically feasible.

The composition of natural gas at the wellhead varies, but the major hydrocarbon present is methane. For example, the methane content of natural gas may vary within the range of from about 40 to 95 volume percent. Other constituents of natural gas may include ethane, propane, butanes, pentanes (and heavier hydrocarbons), hydrogen sulfide, carbon dioxide, helium and nitrogen. Conventional processing of wellhead natural gas yields processed natural gas containing at least a major amount of methane.

Thus there has been interest in developing processes for the conversion of gaseous fuels, including natural gas and methane, to easily transportable, less volatile, value-added products, such as gasoline. Different multi-step processes have been applied to the production of gasoline, typically involving intermediates.

One such intermediate product obtained from methane is synthesis gas. An example of an industrial process proceeding via this intermediate is the Shell middle distillate process, which has been used in Malaysia. This process involves conversion of methane to synthesis gas, followed by the conversion of the synthesis gas to a mixture of hydrocarbons having a large fraction of waxes, followed by cracking of the hydrocarbons to produce a product with the molecular weight distribution of hydrocarbons required for gasoline. Each step is typically carried out with the aid of a catalyst selected for that step. This process has the disadvantage of complexity. Further, the hydrocarbon product tends to be low in aromatics, which are useful increasing the octane of gasoline.

An alternate or additional, intermediate product is methanol. Methanol is produced by the conversion of natural gas or coal to synthesis gas, or direct oxidation from methane. Methanol is then converted to gasoline, typically carried out with the aid of a catalyst. In particular, methanol can be catalytically converted to gasoline boiling range hydrocarbons with a ZSM-5 catalyst, an example of a shape-selective aluminosilicate zeolite catalyst. An industrial example of a methanol to gasoline (MTG) process is the Mobil Oil Process, which has been used in New Zealand and is described, for example, in U.S. Pat. No. 3,894,107 to Butter, et. al. This process has the disadvantage of complexity and its viability appears to be limited to situations in which the cost for supplying an alternate source of gasoline is exceptionally high.

Thus recent efforts at producing gasoline fraction hydrocarbons have focused on one-step catalytic reaction of methane and other light (C₁-C₅) hydrocarbons to form liquid hydrocarbons, such as aromatic liquid hydrocarbons. In particular, as described above, aromatic hydrocarbons are desirable for increasing the octane of gasoline. Further, aromatic hydrocarbons, such as benzene, toluene, ethylbenzene, and xylenes, are an important commodity in the petroleum fuel and petrochemical industries.

Exemplary catalysts disclosed to be active for direct aromatization of various examples of C₁-C₅ hydrocarbons are described in the patents and publication listed in Table 1, each hereby incorporated herein by reference. It can be seen from the table that the catalysts typically include zeolites, aluminosilicates, ZSM-type catalysts, and molecular sieves. Each of these terms is known in the art and they are typically related as follows. The term molecular sieve is typically used to denote a microporous material having cages, channels, or combinations thereof, of molecular dimensions. Molecular sieves are shape selective. The term zeolite typically is used to denote an oxide framework of aluminum and silicon that is an aluminosilicate with a molecular sieve structure. Occasionally, the term zeolite is extended in the art to include any material having the same structural characteristics as an aluminosilicate zeolite. An example of a molecular sieve is a pentasil crystalline aluminosilicate. “Pentasil” is a term used to describe a class of shape-selective molecular sieves. The class of pentasil crystalline aluminosilicates includes, but is not limited to, the following ZSM-type catalysts: ZSM-5, ZSM-8, ZSM-11, ZSM-23 and ZSM-35.

TABLE 1
Catalytic aromatization

| Publication | Feed | Catalyst |
|---|---|---|
| U.S. Pat. No. 4,497,970 | aliphatic or alicyclic | Aluminosilicate zeolite with a silica to |
| | hydrocarbons, particularly | alumina ratio greater than 12:1 |
| | methane, ethane and/or ethylene | |
| U.S. Pat. No. 4,724,271 | C₂ to C₅ aliphatic hydrocarbons | alumina containing phosphorous, |
| | | gallium, and a crystalline |
| | | aliminosilicate having a silica to |
| | | alumina ration of at least 12 |
| U.S. Pat. No. 5,012,029 | methane | crystalline zeolite catalyst |
| U.S. Pat. No. 5,015,798 | methane | intermediate pore size zeolite |
| U.S. Pat. No. 5,026,937 | methane | gallium and a ZSM-type zeolite |
| | | incorporated in a phosphorous- |
| | | containing alumina |
| U.S. Pat. No. 5,936,135 | lower alkane or mixture of lower | bifunctional pentasil zeolite |
| | alkanes | |
| U.S. Pat. No. 5,672,796 | C₃ to C₆ saturated hydrocarbons | partially sulfided Pt/Re loaded, |
| | | essentially hydrogen-form, crystalline |
| | | aluminosilicate molecular sieve |
| | | exhibiting the MFI structure |
| EP 0228267 | methane-rich hydrocarbon | gallium loaded zeolite containing a |
| | feedstock | group VIIB metal |

Despite continuing investigations of catalysts and methods useful for aromatization of light hydrocarbons, challenges remain in economical and feasible industrial implementation of systems and methods of aromatization of light hydrocarbons. For example, it is known that the upgrading of methane to higher hydrocarbons involves endothermic reaction steps. Further, the overall reaction to produce aromatics is very endothermic. The cooling effect caused by the reaction lowers the reaction temperature enough to greatly reduce the reaction rate if make-up heat is not provided in some manner. Thus, it is desirable to provide heat to the reaction. Various methods contemplated for supplying heat for the aromatization of methane are disclosed in U.S. Pat. No. 5,026,937, which is incorporated by reference herein.

One method of providing the heat of reaction is the use of a heat-exchange fluid flowing through the reaction zone, which provides indirect heat to the catalyst in a reaction zone. This method has the disadvantage of inefficient heat exchange and disruption of catalyst flow in non-fixed bed reactors

Alternatively, heat is supplied to the reaction by using more than one reaction zone in sequence, in combination with reheating the reactants provided between the reaction zones. In this interstage reheating the reactant effluent of a first bed of catalyst is heated to the desired inlet temperature of a second downstream bed of catalyst.

One method of interstage reheating includes the use of indirect heat exchange. In this method, the effluent from an upstream reaction zone is passed through a heat exchanger in which the effluent is heated and then passed into a subsequent reactor. The high temperature fluid employed in this indirect heat exchange method may be high temperature steam, combustion gases, a high temperature process stream or other readily available high temperature fluids. This method of interstage heating does not dilute the reactants but does impose some pressure drop in the system and can expose the reactants to undesirably high temperatures.

Another method of interstage heating is the oxidative reheat method. This involves the admixture of a controlled amount of oxygen into the reactants and the selective oxidation of hydrogen. The oxidation, as disclosed in U.S. Pat. No. 5,026,937, is preferably accomplished in the presence of a catalyst that selectively promotes the oxidation of hydrogen as compared to the destructive combustion or oxidation of the more valuable feed and product hydrocarbons. This method of interstage heating has the disadvantage that a second catalyst is used, thus introducing added complexity and cost.

Notwithstanding the foregoing patents and teachings, there remains a need for an efficient and economical system and process for the provision of the heat of reaction to sustain direct catalytic conversion of light hydrocarbons to aromatic hydrocarbons.

### SUMMARY OF THE INVENTION

The present invention features a system and method for circulating catalyst between a reactor system and a regenerator system. A circulating catalyst system includes a reactor system, a regenerator system, and a distribution unit. The reactor system and the regenerator system are adapted to exchange catalyst there between. The regeneration system preferably includes a regeneration zone adapted for the contact of catalyst with a regeneration gas. The system and method are adapted so that one or more regeneration gas may each contact an amount of the catalyst. The distribution unit is adapted to control the percentage of catalyst contacting each regeneration gas. The distribution unit is adapted to select the percentages so as to maintain the reactor system and regeneration system under a heat balance regime. Heat is preferably transferred from the regenerator system to the reactor system by an exchange of catalyst but may also or alternatively be transferred using another heat transfer medium.

The catalyst is preferably a catalyst active for the conversion of a light hydrocarbon to an aromatic hydrocarbon. Further, the catalyst is preferably one that is subject to formation of coke deposited on the catalyst during the reaction. Thus, regeneration of the catalyst preferably includes substantially removing the coke. More particularly, the catalyst preferably includes an aluminosilicate molecular sieve, more preferably a ZSM-5 molecular sieve, most preferably with a silica to alimina ratio of 50:1. Further, the catalyst preferably includes molybdenum loaded on the aluminosilicate. The reactor system preferably includes a fluidized bed of catalyst. The fluidized bed is preferably maintained in a riser reactor.

The regenerator system preferably includes a second fluidized bed of catalyst. The fluidized bed is preferably maintained in a bubbling bed reactor.

The regeneration gas is preferably selected from the group consisting of an oxygen-containing gas, hydrogen gas, and steam. It will be understood that throughout the specification oxygen will be used to refer to any oxygen-containing gas, which may be any one or a combination of oxygen gas, air, and the like. Further, it will be understood that throughout the specification the terms hydrogen and hydrogen gas will be used interchangably. Still further, it will be understood that steam includes any gas containing gaseous water.

In one embodiment, the regeneration system includes a regeneration zone for contacting any of the regeneration gases with the catalyst. Different regeneration gases may be fed to the regeneration zone at different times. Thus, in this embodiment, the present system and method for catalyst circulation are adapted for regenerating different portions of catalyst each by different respective regeneration gases. The different portions may be physically intermingled.

In an alternative embodiment, the regeneration system includes any one or combination of an oxygen regeneration zone, a hydrogen regeneration zone, and a steam regeneration zone. Different amounts of catalyst may be fed to each regeneration zone. Thus, in this alternative embodiment also, the present system and method for catalyst circulation are adapted for regenerating different portions of catalyst each by different respective regeneration gases.

The distribution unit preferably includes one or more valves adapted for controlling passage of either catalyst or a regeneration gas to the regenerator system. The distribution unit may further include a microprocessor for controlling any valves or other equivalent elements of the distribution unit.

One aspect of the present invention features a circulating catalyst system that includes a catalyst convertible between active catalyst active for the aromatization of a light hydrocarbon and spent catalyst, a reactor system comprising a reaction zone in which the catalyst is turned from the active catalyst into the spent catalyst, a regenerator system including a regeneration zone that includes a regeneration zone in which the spent catalyst contacts a regeneration gas, and a distributor unit connected to the regenerator system and controling the amount of spent catalyst entering the regenerator system.

Another aspect of the present invention features a method of supplying heat to a reactor system that includes passing spent catalyst from the reactor system to a regenerator system, with the spent catalyst comprising an active catalyst for the conversion of a light hydrocarbon to an aromatic hydrocarbon and having coke deposited on the active catalyst, removing coke from the spent catalyst in the regenerator system to produce regenerated active catalyst such that heat is generated and stored in said catalyst, and passing the regenerated active catalyst back to the reactor system.

Still another aspect of the present invention features a method for aromatization of a methane-containing feed, the method including contacting a feed stream comprising methane with an active catalyst in a reaction zone under conversion promoting conditions sufficient to produce a product stream, where the active catalyst is deactivated to form spent catalyst. The deactivation may be by coking. The method further includes contacting a portion of the spent catalyst with a regeneration gas stream in a regeneration zone under regeneration promoting conditions sufficient to substantially regenerate the spent catalyst to reform the active catalyst; and cycling between the two steps described above.

The present system and method for circulating catalyst are adapted for simultaneous regeneration of catalyst and heat generation, while controlling the amount of heat generated to balance the heat taken up during conversion of light hydrocarbons to aromatics.

Thus, the present invention comprises a combination of features and advantages which enable it to overcome various problems of prior devices. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a schematic drawing of a circulating catalyst system according to a preferred embodiment of the present invention;

FIG. 2 is a schematic drawing of an alternative circulating catalyst system according to an alternative embodiment of the present invention.

### DETAILED DESCRIPTION OF A PREFERRED EMBODIMENT

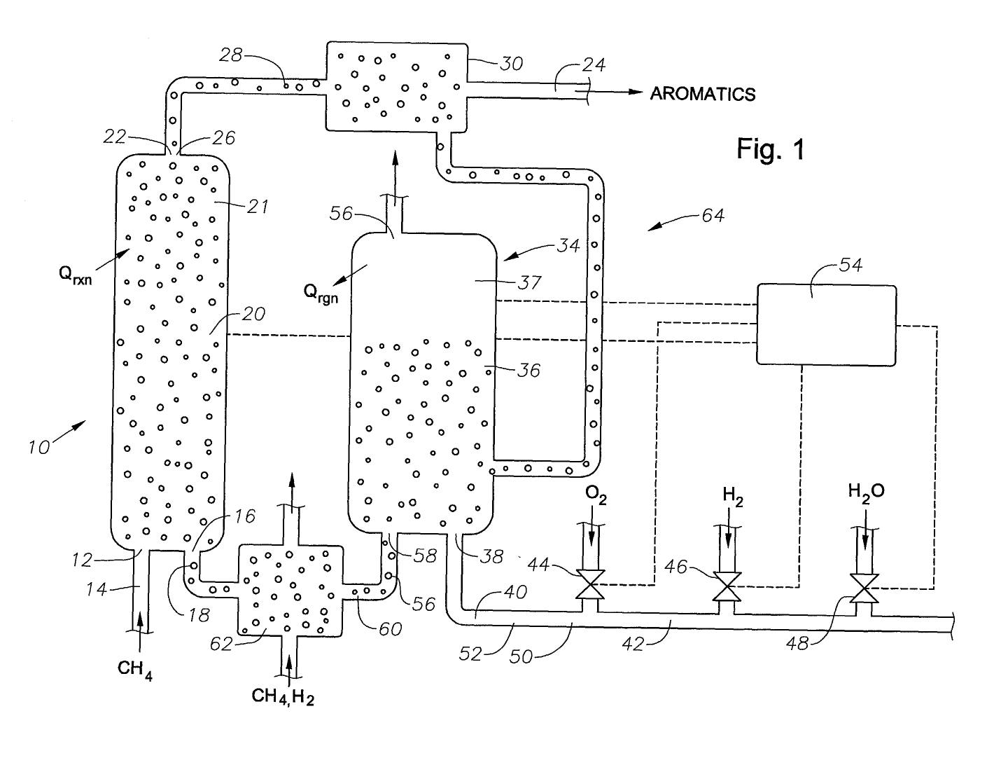

Referring to FIG. 1, a reactor system 10 includes a feed inlet 12 for receiving a feed stream 14. Feed stream 14 includes a light hydrocarbon compound. Light hydrocarbons, as used herein, include hydrocarbon compounds having 1 to 5 carbons. Representative light hydrocarbons include methane, ethane, propane, propylene, n-butane, isobutane, n-butene, isobutene, pentane, pentene, and the like. A preferred feed stream includes a mixture derived from natural gas according to methods known to one of ordinary skill in the art. As used herein, it is more preferred that the feed stream contains a molar concentration of at least 40 percent methane, and it is highly preferred that the feed stream contains at least 50 mole percent methane. Alternatively, the feed stream can be a stream of essentially pure methane, although even a pure stream of gas is likely to contain some small amount, up to 0.5 mole percent of impurities. The impurities may be nitrogen or other inorganic species. The essentially pure methane feed stream can also contain additional light hydrocarbons having chain lengths of up to 5, due to the less than perfect separations used in commercial scale processes.

Still referring to FIG. 1, reactor system 10 further includes a catalyst inlet 16 for receiving active catalyst 18. Catalyst 18 is preferably in the form of particles, more preferably particles suitable for fluidization. Catalyst 18 may be any catalyst useful for the aromatization of a light hydrocarbon, including in particular catalyst material that forms coke as the aromatization reaction proceeds. Coke, as is known in the art, is solid carbon deposited on the catalyst. Catalyst 18 is preferably a catalyst as disclosed in commonly assigned U.S. Provisional Application Serial No. 60/221,082, entitled “Catalyst and Process for Aromatic Hydro Carbons Production from Methane” which is incorporated herein by reference. Exemplary catalysts, as described therein, tend to form coke while catalyzing the aromatization of a methane containing feed stream. More particularly, catalyst 18 is preferably a shape-selective molecular sieve, more preferably a ZSM-5 crystalline aluminosilicate, and still more preferably a ZSM-5 crystalline aluminosilicate having a silica-to-alumina ratio of 50:1.

Catalyst 18 preferably further contains a metal component loaded on the aluminosilicate. The preferred metal component is molybdenum or a molybdenum compound. The final conversion catalyst preferably contains less than 10 wt. percent metal as measured on an elemental analysis basis. It is preferred that the final catalyst contains from about 0.5 to about 4.0 wt. percent total metal component. A highly preferred concentration for molybdenum on the final conversion catalyst is from about 0.5 to about 2.0 wt. percent. The metal component may be loaded, or deposited, on the zeolite by any suitable method, such as any of the following methods. The catalytically active metal may be added by the incipient wetness impregnation of a water-soluble metal salt, such as the ammonium heptamolybdate. Another suitable method is the direct vaporization of the catalytically active metal, such as molybdenum oxide, onto the crystalline aluminosilicate. Other methods as are known in the art may also be used. The catalytically active metal is preferably uniformly distributed throughout the entire network of the final methane conversion catalyst.

In addition, catalyst 18 may include an amorphous silica layer for improving shape selectivity by passivating the external surface of the support which contains acidic sites, coke precursor sites, and any non-shape selective molybdenum catalyst. The amorphous silica-passivating layer is not believed to affect the accessibility of catalyst pores. An amorphous silica layer may be obtained by means of well-known techniques, such chemical vapor deposition or chemical liquid deposition of silicon alkoxides, most preferably tetraethoxysilane. The crystalline aluminosilicate composition that is formed can be separated and recovered by filtration with aqueous washing. Typically, calcination at temperatures ranging from about 350° C. to about 600° C. and preferably from about 450° C. to about 550° C. is preferred to remove organic compounds on the surface of the molecular sieve.

Still referring to FIG. 1, reactor system 10 additionally includes a reaction zone 20 for contacting feed stream 14 with catalyst 18. Reactor system 10 may include multiple reaction zones (not shown). Further, reaction zone 20 may include a single reactor 21 or several separate reactors in series or in parallel or combination thereof (not shown). Catalyst 18 may be maintained as an immobile or fixed bed. Alternatively, catalyst 18 may be maintained as a moving bed. Still alternatively and preferably, catalyst 18 is maintained as a fluidized bed. A fluidized bed reactor system has the advantage of allowing continuous removal of catalyst from the reaction zone, with the withdrawn catalyst being replaced by fresh or regenerated catalyst. Reactor system 10 may be any conventional circulating fluidized reactor system, such as have found widespread use in fluidized catalytic cracking. Conventional fluidized beds include bubbling beds, turbulent fluidized beds, fast fluidized beds, cocurrent pneumatic transport beds, and the like. In a preferred configuration, reaction zone 20 includes a riser reactor. Riser reactors are known in the art of fluidized reactors and include an extended riser feeding the reactor, so that up to 90% or more of reaction occurs in the riser. Riser reactors are particularly useful in combination with ZSM-type zeolite catalysts, due to their high activity.

Still referring to FIG. 1, reactor system 10 includes a product outlet 22, which receives a product stream 24 from reaction zone 20. Product stream 24 preferably includes an aromatic hydrocarbon or a mixture of aromatic hydrocarbons. Aromatic hydrocarbons include benzene, toluene, zylene, naphthalene, and the like. Product stream 24 is preferably suitable for processing for inclusion in a synthetic fuel or for adding to a fuel to increase the octane of the fuel.

Still referring to FIG. 1, reactor system 10 also includes a catalyst outlet 26 that receives spent catalyst 28 from reaction zone 20. Spent catalyst 28 results from the activity of active catalyst 18 as it passes though reaction zone 20. Spent catalyst 28 is preferably suitable for regeneration into active catalyst 18. More preferably, spent catalyst 28 can be regenerated into active catalyst 18 in a process that releases heat. In one preferred embodiment, spent catalyst 28 includes coke (carbon) deposited on catalyst 18.

Still referring to FIG. 1, catalyst outlet 26 connects to a catalyst stripper 30, which removes any components present in reaction zone 20, which may be retained with spent catalyst 28, such as feed components and product components. Catalyst stripper 30 may be any conventional stripper capable of stripping fluids and hydrocarbons from a solid material.

Still referring to FIG. 1, catalyst stripper 30 connects to catalyst inlet 32 forming a part of regeneration system 34. The use of gas-solid disengagement by conventional equipment could be performed on stream 28 prior to the stripper. Such conventional equipment includes settling devices and cyclones. Regeneration system 34 additionally includes a regeneration zone 36 for contacting a regeneration stream 40 with catalyst 28. Regeneration system 34 may include multiple regeneration zones (not shown). Further, regeneration zone 36 may include a single reactor 37 or several separate reactors in series or in parallel or combination thereof (not shown). Within zone 36, catalyst 28 may be maintained as an immobile or fixed bed. Alternatively catalyst 28 may be maintained as a moving bed. Still alternatively, and preferably, catalyst 28 is maintained as a fluidized bed. It is preferred that reaction zone 36 include a fluidized bed of catalyst, such as described above. A fluidized bed regenerator system has the advantage of allowing continuous removal of regenerated catalyst from the regeneration zone, with the withdrawn catalyst being replaced by spent catalyst from reactor system 10. Regenerator system 36 may be any conventional circulating fluidized regenerator system, such as have found widespread use in fluidized catalytic cracking. Regeneration zone 36 preferably includes a fluidized bed in the form of a bubbling bed.

Still referring to FIG. 1, regenerator system 34 further includes a regeneration gas inlet 38 through which regeneration stream 40 is fed into regeneration zone 36. Regeneration stream 40 includes a regeneration gas, preferably containing a gas capable of regenerating spent catalyst 28, preferably a gas capable of de-coking, or reducing the amount of coke deposited on, spent catalyst 28. Regeneration gases suitable for removing coke from catalysts include oxygen-containing gases, such as oxygen or air, hydrogen, and steam. Thus, regeneration stream 34 preferably includes a gas chosen from an oxygen-containing gas, hydrogen, and steam or mixtures thereof. A mixture including oxygen and hydrogen preferably includes them in amounts and under conditions such that the mixture is nonexplosive.

Still referring to FIG. 1, reactor system 10 further includes a regeneration distribution unit 42. Distribution unit 42 preferably includes valves 44, 46, and 48 for controlling the percentages of catalyst regenerated by an oxygen-containing gas, hydrogen, and steam, respectively. Distribution unit 44 preferably further includes an outlet 50 connected to a line 52 that connects to regeneration gas inlet 38. It is understood that the presence of the respective regeneration gases in any lines is controlled to eliminate any possible undesired contact between the respective regeneration gases. Distribution unit 42 may also include a microprocessor 54 for automated control of the percentages of catalyst regenerated by the different regeneration gases. In one embodiment, microprocessor 54 receives inputs such as temperature from reaction zone 20, regeneration zone 36, or both zones.

Regeneration zone 36 includes a byproduct outlet 55. Byproducts vary with the regeneration gas and are known to one of ordinary skill in the art. For example, hydrogen regeneration may produce methane as a byproduct.

Still referring to FIG. 1, regenerator system 30 preferably includes a catalyst outlet 58 for passing regenerated catalyst 56 from regeneration zone 36. Regenerated catalyst 56 is obtained from spent catalyst 28 by passing it though regeneration zone 30. Regenerated catalyst 56 is preferably substantially returned to the form of active catalyst 18. Active catalyst 18 may optionally include fresh catalyst. In a preferred embodiment, regenerated catalyst 56 includes only a minor, essentially zero, amount of coke deposited on spent catalyst 28. Thus, regenerated catalyst 56 preferably substantially lacks carbon deposited on spent catalyst 28.

Still referring to FIG. 1, catalyst outlet 58 connects to a catalyst transfer system 60 that connects to catalyst inlet 16 forming a part of a reactor system 10. Catalyst transfer system 60 may optionally include a reduction vessel 62 for increasing the activity of regenerated catalyst 56. Reduction vessels are known in the art and may include catalyst in a fluidized bed. Reduction vessel 62 is adapted for contacting regenerated catalyst 56 with a reducing gas stream including hydrogen and methane or mixtures thereof.

Thus, reactor system 10 is connected to regenerator system 36. In particular, reactor system 10 and regenerator system 36 form a circulating catalyst system 64.

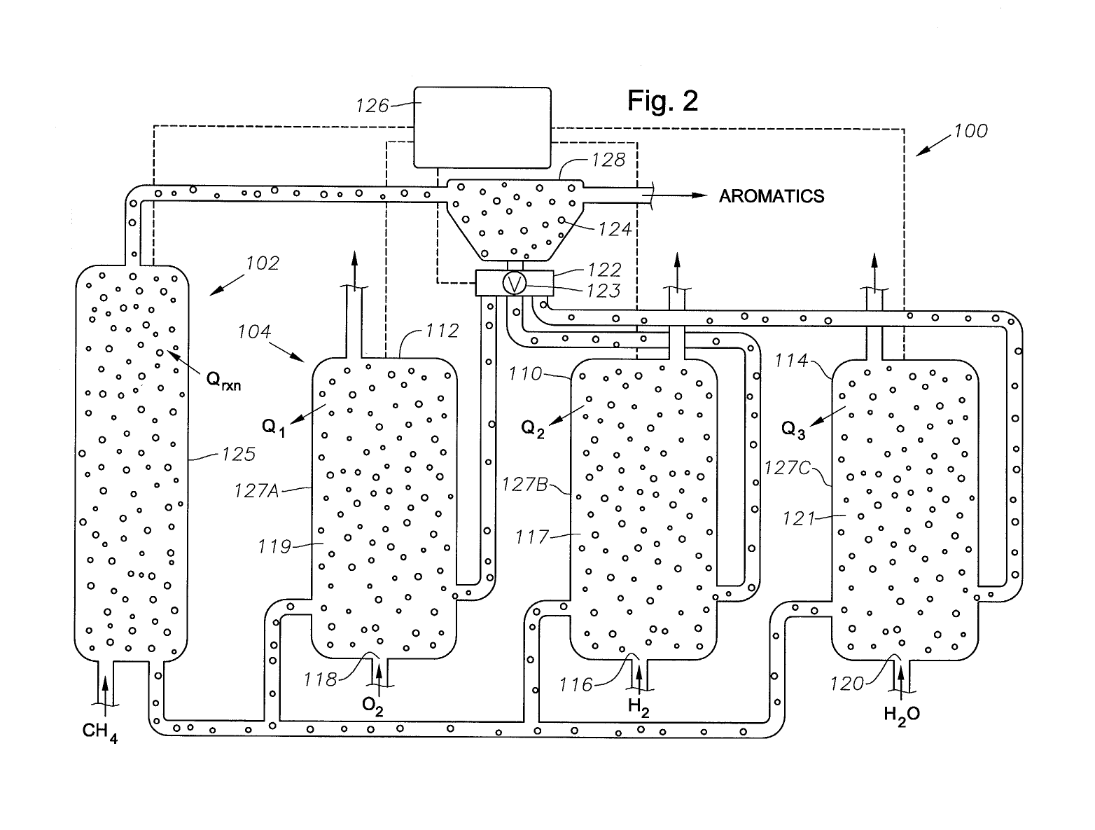

Referring now to FIG. 2, an alternative preferred embodiment of circulating catalyst system 100 includes a reaction system 102 and a regenerator system 104. Regenerator system 104 includes a hydrogen regenerator 110, an oxygen regenerator 112, and a steam regenerator zone 114. Hydrogen regenerator 110 includes hydrogen inlet 116 for receiving hydrogen gas and a hydrogen regeneration zone 117. Oxygen regenerator 112 includes an oxygen inlet 118 for receiving an oxygen-containing gas and an oxygen regeneration zone 119. Steam regenerator 114 includes steam inlet 120 for receiving steam and a steam regeneration zone 121. Circulating catalyst system 100 preferably includes a regeneration distribution unit 122 for selecting the respective percentages of spent catalyst 124 passed to hydrogen regenerator 110, oxygen regenerator 112, and steam regenerator 114 and thus regenerated by an oxygen-containing gas, hydrogen, and stream respectively. Distribution unit 122 may include a distribution valve 123. Distribution unit 122 may also include a microprocessor 126 for automated control of distribution valve 123 and thus control of the percentages of catalyst regenerated by the different regeneration gases. In one embodiment, microprocessor 126 receives inputs such as temperature from reaction zone 125, any regeneration zone 127, or combination thereof. Regeneration distribution unit 122 is connected to a stripper 128 and to each of the regenerators 110, 112, and 114. It will be understood that a regenerator system may include any one or combination of a hydrogen regenerator, and oxygen regenerator, and a steam regenerator.

Referring to FIGS. 1 and 2, it will be understood that a fluidized bed may be a lean-phase bed with entrainment of solids or a dense-phase bed with an upper surface to the bed. A bubbling bed is an example of a dense-phase bed. Further, a fluidized bed may operate either in upflow cocurrent mode, downflow countercurrent mode, or crosscurrent mode. Thus, as will be understood by one of skill in the art, the arrangement of reactor and regenerator inlets and outlets depicted schematically in FIGS. 1 and 2 is exemplary only and not limiting. Further, it is understood that either of the reactor system or regenerator system may include one or more cyclones, heat coils, disengaging zones and other elements as are known in the art of fluidized systems.

Referring to FIGS. 1 and 2, a selection of regeneration gases, as controlled by a distribution unit such as distribution unit 50, 122, allows the amount of heat generated by the regenerator system to be selected by selecting the relative percentages of the spent catalyst regenerated by each regeneration gas, as further described below. In particular, referring to FIG. 1, the regenerator heat Q_{rgn} preferably balances the reactor heat Qᵣₓₙ. Likewise, referring to FIG. 2, the combination of regenerator heat Q_{rgn}=Q₁+Q₂+Q₃ preferably balances the reactor heat Qᵣₓₙ.

A circulating catalyst system that includes a reactor system coupled to a regeneration system, such as described above, operates as a thermodynamic system. The reactor system includes a reaction zone in which a feed stream containing a reactant contacts a catalyst and is converted in an endothermic reaction process into an exit stream containing a product. Likewise, the regeneration system includes a regeneration zone, in which a regeneration stream contacts the catalyst and is converted in an exothermic reaction process into an effluent stream. The regeneration system is coupled to the reaction system by the exchange of catalyst. Further, the regeneration system is preferably coupled to the reaction system by an exchange of heat. The heat is preferably exchanged through the exchange of catalyst.

The circulating catalyst system preferably operates as a substantially isolated thermodynamic system in which the reaction system and the regeneration system operate under energy balance conditions. More preferably, the reaction system and the regeneration system operate under heat balance conditions. In particular, preferably neither the reactor system nor the regeneration system performs work. Thus, a balance in heat preferably is also a balance in energy. Further, for a thermodynamic system undergoing a change of state at constant pressure, when there is no non-PV work, the heat associated with that change equals the change in enthalpy. Thus, for such a system, a balance in enthalpy is also a balance in heat.

The endothermic reaction process may include a single reaction or a plurality of reactions. Individual reactions may be endothermic, that is have a positive reaction enthalpy, or exothermic, that is have a negative reaction enthalpy, so long as the overall reaction process is endothermic. The endothermic reaction process preferably includes the catalytic aromatization of a light hydrocarbon to form an aromatic compound. Each aromatization reaction is preferably non-oxidative. The endothermic reaction process also includes catalyst deactivation, such as by deposition of coke on the aromatization catalyst.

Likewise, the exothermic reaction process may include a single reaction or a plurality of reactions. The individual reactions may be endothermic or exothermic, so long as the overall regeneration process is exothermic. The exothermic regeneration process preferably includes the regeneration of the catalyst through removal of coke. Thus, the exothermic regeneration process may include oxygen regeneration, hydrogen regeneration, and steam regeneration.

According to a preferred embodiment, the distribution unit is used to select the percentages of catalyst regenerated by any of an oxygen-containing gas, hydrogen, and steam. Thus, the heat released by the regeneration process may be selected according to the thermal needs of the reactor system. In particular, in a preferred embodiment the percentages are selected such that the reactor system and regenerator system operate under heat balance conditions. In a more preferred embodiment the percentages are selected such that the reactor system and the regenerator system operate under enthalpy balance conditions.

Exemplary Operation

It is understood that a method of operating the circulating reactor may include selection of the conditions of operation. The feed stream preferably has a temperature from about 50° C. to about 800° C. The feed stream preferably contacts the catalyst under conditions of a pressure of about 1 atmosphere to about 40 atmospheres and a temperature of about 600° C. to about 900° C. The product stream preferably has a temperature from about 600° C. to about 900° C. The catalyst preferably is transferred from reactor system to regenerator system without a change in temperature and is transferred to the regenerator system. A regeneration stream preferably has a temperature of from about 700° C. to about 1200° C.

### EXAMPLE

In the following example, a model calculation of enthalpy balance was performed for an exemplary thermodynamic system. An exemplary reaction process included reactions a and b listed in Table 2. An exemplary regeneration process included at least one of the reactions c, d, and e listed in Table 2. For the model calculation, it will be understood that, unless indicated otherwise, all species were present in their standard states.

The standard molar reaction enthalpy ΔHₘ° (j) for reaction j was computed from the standard enthapies of formation ΔH_{T,i} of each reaction and product specie i and from the reaction coefficients, as indicated in Table 2.

TABLE 2
Enthalpy changes from temperature dependent heats of formation

| reaction | | enthalpy change at temperature T |
|---|---|---|
| CH₄→⅙C₆H₆ + 3/2H₂ | a | ΔHₐ°(T) = −ΔH_{T,CH4}°+(⅙)ΔH_{T,C6H6}°+(3/2)ΔH_{TH2}° |
| CH₄→C + 2H₂ | b | ΔH_{b}°(T) = −ΔH_{T,CH4}°+ΔH_{T,C}°+2ΔH_{T,H2}° |
| C + O₂→CO₂ | c | ΔH_{c}°(T) = ΔH_{T,C}°−ΔH_{T,O2}°+ΔH_{T,CO2}° |
| C + 2H₂→CH₄ | d | ΔH_{d}°(T) = −ΔH_{T,C}°−2ΔH_{T,H2}°+ΔH_{T,CH4}° |
| C + H₂O→CO + H₂ | e | ΔHₑ°(T) = −ΔH_{T,C}°−ΔH_{T,H20}°+ΔH_{T,H2}° |

The standard enthalpy of formation at temperature T for each specie i was determined, from tabulated values for thermodynamic parameters. In particular, the following relationship was used:

$$Δ {H}_{T , i} = Δ {H}_{f , i} + {\int }_{298.15}^{T} {C}_{p , i} ( T ) ⅆ T , \qquad \text{Eq. 1}$$

where ΔH_{f,i} is the standard enthaply of formation for specie i at or room temperature, that is 25 C (298.15), and C_{p,i}(T) is the constant pressure heat capacity at temperature T. C_{p,i}(T) was estimated from

C_{p,i}=R(Aᵢ+BₗT+CᵢT²+DₗT⁻²), Eq. 2

where R is given by 8.314 J/mol-K, T is in degrees Kelvin. Values used for ΔH_{f,i}°, Aᵢ, Bᵢ, Cᵢ, and Dᵢ for each species i are listed in Table 3.

TABLE 3
Thermodynamic parameters

| Species i | ΔH_{f,i}° (J/mol) | Aᵢ | Bᵢ × 10³ | Cᵢ × 10⁶ | Dᵢ × 10⁻⁵ |
|---|---|---|---|---|---|
| CH₄ | −74.847 | 1.702 | 9.081 | −2.164 | 0 |
| C₆H₆ | 82.927 | −0.206 | 39.064 | −13.301 | 0 |
| H₂ | 0 | 3.249 | 0.422 | 0 | 0.083 |
| C(graphite) | 0 | 1.771 | 0.771 | 0 | −0.867 |
| O₂ | 0 | 3.639 | 0.506 | 0 | −0.227 |
| CO₂ | −393.51 | 5.457 | 1.045 | 0 | −1.157 |
| CO | −110.53 | 3.376 | 0.557 | 0 | −0.431 |

The formal reaction for the reaction process is

Sₐ(reaction a)+S_{b}(reaction b),

where Sₐ is the percentage of methane converted to benzene and S_{b} is the percentage of methane converted to coke. Each is termed a selectivity. Thus the reaction process molar enthalpy,

ΔHᵣₓₙ=η[SₐΔHₐ°(Tᵣₓₙ)+S_{b}ΔH_{b}°(Tᵣₓₙ)] Eq. 3

where η is the percentage of methane converted with respect to methane feed. The formal reaction for the regeneration process is

χ_{c}(reaction c)+χ_{d}(reaction d)+χₑ(reaction e),

where χ_{c} is the percentage of catalyst regenerated by oxygen, χ_{d} is the percentage of catalyst regenerated by hydrogen, and χₑ is the percentage of catalyst regenerated by steam. Each is herein termed a regeneration percentage. Thus the regeneration process molar enthalpy, with respect to methane is feed

ΔH_{rgn}=ηS_{b}[χ_{c}ΔH_{c}°(T_{c})+χ_{d}ΔH_{d}°(T_{d})+χₑΔHₑ°(Tₑ)] Eq. 4

The total enthalpy contributed by the combination of the formal reactions of reaction and regeneration is

ΔHᵢₛₒ=ΔHᵣₓₙ(Tᵣₓₙ)+ΔH_{rgn}(T_{rgn}) Eq. 5

where the subscript iso denotes isothermal reactors and isothermal regenerator. In the absence of temperature, pressure, and matter variations that may also effect the enthalpy change, the condition of enthalpy balance between the reactor system and the regeneration system is that ΔHᵢₛₒ is substantially zero.

The enthalpy change for the circulating catalyst system may include other contributions, such as for a temperature gradient in a reactor. Under some conditions it may be desired to allow some temperature change to occur in the reactor. Thus, the condition of enthalpy balance for the reactor system and regeneration system may include terms accounting for other effects, such as a change of temperature or a temperature gradient. Adjustments for these effects may be made by known methods within the understanding of one or ordinary skill in the art.

In particular, the model calculation incorporated possible non-isothermal effects. The enthalpy change for a non-isothermal reactor system is

ΔH_{RXS}=ΔHᵣₓₙ(Tᵣₓₙ)+H₂(Tₚ)−H₁(T_{F}) Eq. 6

where T_{F} is the feed temperature and Tₚ is the product stream temperature. Likewise, the enthalpy change for a non-isothermal regenerator system is

ΔH_{RGS}=ΔHᵣₓₙ(Tᵣₓₙ)+H₄(T_{E})−H₃(T_{R}) Eq. 7

where T_{R} is the regeneration stream temperature and T_{E} is the effluent stream temperature. H₁, H₂, H₃, and H₄ are molar enthalpies with respect to methane feed. They were each computed according to

$${H}_{j} ( T ) = {\sum }_{i ∈ streamj} {n}_{i} Δ {H}_{T , i} , \qquad \text{Eq. 8}$$

where j is any of 1, 2, 3, or 4, T correspondingly represents any of T_{X}, T_{P}, T_{R}, and T_{E} respectively, ni is the molar amount of species i contained in the corresponding stream, and ΔH_{T,i} is computed according to Eq. 1. Each nᵢ used in implementing Eq. 8 was relative to the moles of methane in the feed stream. The nᵢ were determined according to mass balance using the reaction coefficients in Table 2, the selectivities, the methane conversion, and the regeneration percentages.

Thus for a system in which T_{F} and T_{P} may differ from Tᵣₓₙ and T_{R} and T_{E} may differ from T_{rgn}, the enthalpy change for the combined system of reactor system and regenerator system is:

ΔH=ΔH_{RXS}+ΔH_{RGS}

Thus, in this system, the condition of enthalpy balance is ΔH is substantially zero.

Each of these standard enthalpy changes was used in modeling the enthalpy change for each of the reactor system and regenerator system. The results are given in Table 4, for a methane conversion of 0.4, a methane feed temperature of 750° C. (T_{F}), a product exit temperature of 100° C. (T_{P}), and the regeneration feed (T_{R}) and effluent temperatures (T_{E}) being the same as the regeneration temperature. The molar enthalpy changes are given per mole of methane feed.

TABLE 4
Enthalpy balance

| | | Tᵣₓₙ | T_{rgn} | | | | ΔH_{RXS} | ΔH_{RGS} |
|---|---|---|---|---|---|---|---|---|
| Sₐ | S_{b} | (° C.) | (° C.) | χ_{c} | χ_{d} | χₑ | (kJ/mol) | (kJ/mol) |
| .091 | .909 | 750 | 750 | 0 | 1 | 0 | 33.8 | −33.8 |
| .395 | .605 | 750 | 750 | 1 | 0 | 0 | 95.6 | −95.6 |

These calculations are exemplary and not limiting. It is believed that the regeneration percentages may be tuned to balance the enthalpy changes of regenerator system and reactor system for any value of the coke selectivity in the range of 0.25 to 0.95. Further, a similar procedure may be used for alternative light hydrocarbon reactants and for alternative aromatic products.

It will be appreciated by one of skill in the art that the flow rates may be adjusted, as may the temperatures, Tᵣₓₙ, T_{rgn}, T_{E}, T_{R}, T_{F}, and T_{R} to meet the target heat balance conditions. Further, ΔH_{RXS} and ΔH_{RGS} may each contain terms from a temperature gradient in any one of an oxygen regenerator, a hydrogen regenerator, and a steam regenerator. It is preferred that the regeneration percentages χ_{c}, χ_{d}, and χₑ are selected according to the selectivities Sₐ and S_{b} to achieve a condition of heat balance between regenerator and reactor. Such selection may be controlled by a microprocessor included in a regeneration distribution unit. For example, it will be appreciated that the microprocessor unit can be programmed to perform the above calculations automatically. Further, the microprocessor unit may receive data from measurements of any one of the above variables.

Further, it will be appreciated that, while the method of heat balance has been described for exemplary reaction and regeneration, with catalyst deactivated by coking, the above-described method may applied for other processes involving a catalyst that is deactivated during an endothermic reaction and is regenerable by an exothermic regeneration process. For example, any reaction process that deposits an elemental species on the catalyst may be coupled with a regeneration process involving exothermic oxidation of the elemental species.

Should the disclosure of any of the patents and publications that are incorporated herein conflict with the present specification to the extent that it might render a term unclear, the present specification shall take precedence.

While preferred embodiments of this invention have been shown and described, modifications thereof can be made by one skilled in the art without departing from the spirit or teaching of this invention. The embodiments described herein are exemplary only and are not limiting. Many variations and modifications of the system and process are possible and are within the scope of the invention. Accordingly, the scope of protection is not limited to the embodiments described herein, but is only limited by the claims that follow, the scope of which shall include all equivalents of the subject matter of the claims.

We claim:

1. A process for aromatization of a methane-containing feed, comprising:
a) contacting a feed stream comprising methane with an active catalyst in a reaction zone so as to produce a product stream, wherein the catalyst is deactivated to form spent catalyst;
b) contacting at least a first portion of the spent catalyst with at least a first regeneration gas stream in at least a first regeneration zone so as to regenerate the first portion to reform at least a first portion of the active catalyst; and
c) cycling between steps (a) and (b).

2. The process according to claim 1 wherein step (b) comprises selecting at least one of the percentage of the first portion of the spent catalyst and the composition of the first regeneration gas.

3. The process according to claim 1 wherein step (c) comprises maintaining conditions of heat balance.

4. The process according to claim 1 wherein the reaction zone comprises a fluidized bed.

5. The process according to claim 1 wherein the reaction zone comprises a riser reactor.

6. The process according to claim 1 wherein the reaction zone comprises a bubbling bed reactor.

7. The process according to claim 1 wherein the regeneration zone comprises a second fluidized bed.

8. The process according to claim 1 wherein the catalyst is deactivated by coking.

9. The process according to claim 1 wherein step (b) further comprises:
contacting a second portion of the spent catalyst with a second regeneration gas stream in the first regeneration zone or in a second regeneration zone so as to regenerate the second portion to reform a second portion of the active catalyst.

10. The process according to claim 9 wherein step (b) comprises selecting at least one of the percentage of the second portion of the spent catalyst and the composition of the second regeneration gas.

11. The process according to claim 9 wherein step (c) comprises maintaining conditions of heat balance.

12. The process according to claim 9 wherein step (b) further comprises
contacting a third portion of the spent catalyst with a third regeneration gas stream in the first regeneration zone or a third regeneration zone so as to regenerate the third portion to reform a third portion of the active catalyst.

13. The process according to claim 12 wherein step (b) comprises selecting at least one of the percentage of the third portion of the spent catalyst and the composition of the third regeneration gas.

14. The process according to claim 12 wherein step (c) comprises maintaining conditions of heat balance.

15. A method of supplying heat to a reactor system, the method comprising:
a) passing spent catalyst from the reactor system to a regeneration system, said spent catalyst comprising an active catalyst for the conversion of a light hydrocarbon to an aromatic hydrocarbon and coke deposited on the active catalyst;
b) removing coke from the spent catalyst in the regeneration system to produce regenerated active catalyst, such that heat is generated and stored in said catalyst; and
c) passing the regenerated active catalyst to the reactor system.

16. The method according to claim 15 wherein the active catalyst comprises a ZSM-5 molecular sieve.

17. The method according to claim 15 wherein the active catalyst comprises molybdenum.

18. The method according to claim 15 wherein the active catalyst comprises an aluminosilicate molecular sieve having a silica-to-alumina ratio of about 50:1.

19. The method according to claim 15 wherein step (b) comprises:
b1) contacting a first percentage of the spent catalyst with a first regeneration gas;
b2) contacting a said second percentage of the spent catalyst with a second regeneration gas; and
b3) selecting said first and second percentages to adjust the amount of heat supplied to the reactor system.

20. The method according to claim 19 wherein said first regeneration gas is a gas selected from the group consisting of an oxygen-containing gas and hydrogen gas.

21. The method according to claim 20 wherein said second regeneration gas is another gas selected from the group consisting of an oxygen-containing gas, hydrogen gas, and steam.

22. A circulating catalyst system, comprising:
a plurality of catalyst particles, said plurality forming a catalyst for the aromatization of a light hydrocarbon, said catalyst convertible between an active catalyst and a spent catalyst and maintained in a fluidized state;
a reactor system comprising a reaction zone in which said catalyst is turned from said active catalyst into said spent catalyst;
a regenerator system comprising a regeneration zone in which said spent catalyst contacts a regeneration gas and is turned from said spent catalyst into said active catalyst.

23. The circulating catalyst system according to claim 22 further comprising:
a distributor unit connected to said regenerator system, said distributor unit controlling the amount of said spent catalyst contacting said regeneration gas.

24. The circulating catalyst system according to claim 23 wherein said distribution unit selects said amount so as to achieve a condition of heat balance between said reactor system and said regenerator system.

25. The circulating catalyst system according to claim 23 wherein said distribution unit comprises a valve.

26. The circulating catalyst system according to claim 25 wherein said distribution unit comprises a microprocessor controlling the operation of said valve.

27. The circulating catalyst system according to claim 23 wherein said distribution unit controls passage of said spent catalyst to said regeneration zone.

28. The circulating catalyst system according to claim 27 wherein said distribution unit selects the amount said spent catalyst entering each of said regeneration zone so as to achieve a condition of heat balance between said reactor system and said regenerator system

29. The circulating catalyst system according to claim 27 wherein said regenerator system further comprises a second regeneration zone in which spent catalyst contacts a second regeneration gas; and wherein said distribution unit further controls passage of spent catalyst to said second regeneration zone.

30. The circulating catalyst system according to claim 29 wherein said distribution unit selects the amounts of said spent catalyst entering each of said regeneration zones to achieve a condition of heat balance between said reactor system and said regenerator system

31. The circulating catalyst system according to claim 29 wherein said regenerator system further comprises a third regeneration zone in which spent catalyst contacts a third regeneration gas; and wherein said distribution unit further controls passage of spent catalyst to said third regeneration zone.

32. The circulating catalyst system according to claim 31 wherein said distribution unit selects the amounts of said spent catalyst entering each of said regeneration zones to achieve a condition of heat balance between said reactor system and said regenerator system.

33. The circulating catalyst system according to claim 23 wherein said distribution unit controls passage of said regeneration gas to said regeneration zone.

34. The circulating catalyst system according to claim 33 wherein said distribution unit selects the amount of said regeneration gas entering said regeneration zone to achieve a condition of heat balance between said reactor system and said regenerator system.

35. The circulating catalyst system according to claim 33 wherein said distribution unit further controls passage of a second regeneration gas to said regeneration zone.

36. The circulating catalyst system according to claim 35 wherein said distribution unit selects the amounts of each of said regeneration gases entering said regeneration zone so as to achieve a condition of heat balance between said reactor system and said regenerator system.

37. The circulating catalyst system according to claim 35 wherein said distribution unit further controls passage of a third regeneration gas to said regeneration zone.

38. The circulating catalyst system according to claim 37 wherein said distribution unit selects the amounts of each of said regeneration gases entering said regeneration zone so as to achieve a condition of heat balance between said reactor system and said regenerator system.
